(19) United States
(12) Patent Application Publication
(10) Pub. No.: US 2026/0306442 A1
(43) Pub. Date: Oct. 8, 2026

(54) METHOD AND PHARMACEUTICAL COMPOSITION FOR TREATING A SLEEP DISORDER

(71) Applicant: OVERSEAS PHARMACEUTICALS, LTD., Guangzhou (CN)

(72) Inventors: Xiaoguang WEN, Guangzhou (CN); Jingya WANG, Guangzhou (CN); Chenliang ZHANG, Guangzhou (CN); Peipei WANG, Guangzhou (CN)

(73) Assignee: OVERSEAS PHARMACEUTICALS, LTD., Guangzhou (CN)

(21) Appl. No.: 19/697,627

(22) Filed: Jun. 4, 2026

Related U.S. Application Data
(63) Continuation-in-part of application No. 18/288,382, filed on Aug. 16, 2022.

(30) Foreign Application Priority Data
Aug. 18, 2021 (CN) 202110950477.6
Jun. 4, 2025 (CN) 202510735345.X

(51) Int. Cl. A61K 31/343; A61K 9/20; A61K 9/28; A61K 31/165; A61K 31/4045

(52) U.S. Cl. CPC A61K 31/343; A61K 9/2009; A61K 9/2013; A61K 9/2018; A61K 9/2027; A61K 9/205; A61K 9/2054; A61K 9/2059; A61K 9/284; A61K 9/2853; A61K 9/2866; A61K 9/2886; A61K 31/165; A61K 31/4045

(57) ABSTRACT

A method for treating a sleep disorder, including: orally administering to a patient in need thereof a modified pharmaceutical composition, where the API of the modified pharmaceutical composition is selected from the group consisting of a melatonin receptor agonist, a GABA receptor agonist, an orexin receptor antagonist, and pharmaceutically acceptable salts, isomers, and active metabolites thereof;
where the melatonin receptor agonist is selected from the group consisting of melatonin, agomelatine, and ramelteon;
where the GABA receptor agonist is selected from the group consisting of zolpidem, zopiclone, zaleplon, triazolam, alprazolam, diazepam, estazolam, clonazepam, and didasinine;
where the orexin receptor antagonist is selected from the group consisting of suvorexant, daridorexant, and lemborexant.

### CROSS REFERENCE TO THE RELATED APPLICATIONS

This application is a continuation application of the U.S. patent application Ser. No. 18/288,382, filed on Oct. 26, 2023. U.S. Ser. No. 18/288,382 is the national phase entry of International Application No. PCT/CN2022/112853, filed on Aug. 16, 2022, which is based upon and claims priority to Chinese Patent Applications No. 202110950477.6, filed on Aug. 18, 2021 and No. 202510735345.X, filed on Jun. 4, 2025; the entire contents of which are incorporated herein by reference.

### TECHNICAL FIELD

The present invention relates to pharmaceutical technology, and particularly to a method for treating a sleep disorder with staged release.

### BACKGROUND

Some drugs such as sleep-regulating drugs need to be rapidly released and take effect quickly after being taken by a patient, but a blood concentration rapidly fluctuates and an adverse reaction rate is high. A preparation technology needs to be developed to ensure that the blood concentration of the drugs in vivo is more stable, a pharmaceutical effect disappears later, and the adverse reaction rate is lower.

### SUMMARY

Based on the problems to be solved in the field, the present disclosure provides a tablet allowing release by two stages of a drug and a preparation method therefor.

The technical solution of the present disclosure is as follows:

In a first aspect, the present invention provides a method for treating insomnia, characterized in that it includes the following steps: orally administering to a patient in need thereof a modified pharmaceutical composition; wherein the API of the modified pharmaceutical composition is selected from the group consisting of a melatonin receptor agonist, a GABA receptor agonist, an orexin receptor antagonist, and pharmaceutically acceptable salts, isomers, and active metabolites thereof;
wherein the melatonin receptor agonist is selected from the group consisting of melatonin, agomelatine, and ramelteon;
wherein the GABA receptor agonist is selected from the group consisting of zolpidem, zopiclone, zaleplon, triazolam, alprazolam, diazepam, estazolam, clonazepam, and didasinine; and
wherein the orexin receptor antagonist is selected from the group consisting of suvorexant, daridorexant, and lemborexant.

In a second aspect, the present invention provides a method for alleviating insomnia, characterized in that it includes the following steps: orally administering to a patient in need thereof a modified pharmaceutical composition;
wherein the API of the modified pharmaceutical composition is selected from the group consisting of a melatonin receptor agonist, a GABA receptor agonist, an orexin receptor antagonist, and pharmaceutically acceptable salts, isomers, and active metabolites thereof;
wherein the melatonin receptor agonist is selected from the group consisting of melatonin, agomelatine, and ramelteon;
wherein the GABA receptor agonist is selected from the group consisting of zolpidem, zopiclone, zaleplon, triazolam, alprazolam, diazepam, estazolam, clonazepam, and didasinine;
wherein the orexin receptor antagonist is selected from the group consisting of suvorexant, daridorexant, and lemborexant.

In a third aspect, the present invention provides a method for alleviating early awakening, characterized in that it includes the following steps: orally administering to a patient in need thereof a modified pharmaceutical composition;
wherein the API of the modified pharmaceutical composition is selected from the group consisting of a melatonin receptor agonist, a GABA receptor agonist, an orexin receptor antagonist, and pharmaceutically acceptable salts, isomers, and active metabolites thereof;
wherein the melatonin receptor agonist includes melatonin, agomelatine, or ramelteon;
wherein the GABA receptor agonist includes zolpidem, zopiclone, zaleplon, triazolam, alprazolam, diazepam, estazolam, clonazepam, or didasinine; and
wherein the orexin receptor antagonist includes suvorexant, daridorexant, or lemborexant.

In a fourth aspect, the present invention provides a method for sleep regulation that reduces hangover effect, characterized in that it includes the following steps: orally administering to a patient in need thereof a modified pharmaceutical composition; wherein the API of the modified pharmaceutical composition is selected from the group consisting of a melatonin receptor agonist, a GABA receptor agonist, an orexin receptor antagonist, and pharmaceutically acceptable salts, isomers, and active metabolites thereof;
wherein the melatonin receptor agonist is selected from the group consisting of melatonin, agomelatine, and ramelteon;
wherein the GABA receptor agonist is selected from the group consisting of zolpidem, zopiclone, zaleplon, triazolam, alprazolam, diazepam, estazolam, clonazepam, and didasinine;
wherein the orexin receptor antagonist is selected from the group consisting of suvorexant, daridorexant, and lemborexant.

The hangover effect refers to symptoms of dizziness, drowsiness, somnolence, fatigue, and disorientation that occur the day after taking chemical hypnotic agents; reducing the hangover effect means causing no or only mild symptoms of drowsiness and somnolence.

In any of the above methods, the dosage of the modified pharmaceutical composition is determined according to the prescribed amount of the API type therein. For example, typically, the dosage of melatonin is 0.5 mg to 10 mg per administration; the dosage of agomelatine is 4 mg to 50 mg per administration; and the dosage of ramelteon is 4 mg to 64 mg per administration.

Preferably, the method according to any one of the preceding aspects, wherein after administration of the modified pharmaceutical composition, the patient's plasma concentration exhibits 2 to 3 peaks, and the time interval between the first peak and the last peak is 0.3 to 10 hours.

Further preferably, the method according to the above, wherein the first peak occurs from 0.1 to 3 hours after administration.

Preferably, the method according to any one of the preceding aspects, wherein the modified pharmaceutical composition is a tablet, each tablet including, from inside to outside in order, a drug-containing delayed-release core, a drug-free enteric coating, and a drug-containing immediate-release shell.

Further preferably, the method according to the above, wherein the drug-free enteric coating has a weight gain relative to the drug-containing delayed-release core of at least 4%, preferably from 4% to 12%, or 5%, 6%, 7%, 8%, and wherein the coating is soluble at a pH above 5, preferably at a pH above 7, such that the delayed-release core begins to release after a lag period of 0.5 to 4 hours following complete release of the drug-containing immediate-release shell, and is fully released within the subsequent 0.1 to 6 hours, preferably within 0.5 to 4 hours.

Further preferably, the method according to the above, wherein a drug-free gastric-soluble coating is further provided between the drug-containing delayed-release core and the drug-free enteric coating, and the weight gain of the drug-free gastric-soluble coating is 2% to 5%, preferably 3% or 4%.

Further preferably, the method according to the above, wherein the film-forming material used in the drug-free enteric coating is selected from the group consisting of Acrylic Resin I, Acrylic Resin II, Acrylic Resin III, cellulose acetate phthalate (CAP), hydroxypropyl methylcellulose phthalate (HPMCP), polyvinyl acetate phthalate (PVAP), cellulose acetate trimellitate (CAT), Eudragit S100, Eudragit L100, methacrylic acid-methyl methacrylate copolymer, and methacrylic acid-ethyl acrylate copolymer, and preferably the film-forming material is a methacrylic acid and ethyl acrylate copolymer.

Further preferably, the method according to the above, wherein the ratio of the amount of active ingredient in the drug-containing immediate-release shell to that in the drug-containing delayed-release core is from 1:1 to 3:1.

Further preferably, the method according to the above, wherein the drug-containing delayed-release core is an immediate-release formulation; and wherein in the drug-containing delayed-release core, the weight percentages of the excipients are as follows: filler: 60-90%; disintegrant: 2-10%, preferably 4-8%; binder: 2-6%, preferably 2.5-5%; antioxidant: 0.01-1%, preferably 0.05-1%; lubricant/glidant: 0.5-3%, preferably 0.5-1.5%; wherein the filler and the antioxidant are intragranular excipients, the lubricant/glidant is an extragranular excipient, and the disintegrant can be either an intragranular excipient or an extragranular excipient.

Further preferably, the method according to the above, wherein the drug-containing delayed-release core is a sustained-release formulation; and wherein in the drug-containing delayed-release core, the weight percentages of the excipients are as follows: filler: 60-90%; binder: 2-6%, preferably 2.5-5%; sustained-release material: 4-25%, preferably 5-20%; antioxidant: 0.01-1%, preferably 0.05-1%; lubricant/glidant: 0.5-3%, preferably 0.5-1.5%; wherein the filler, the binder, and the antioxidant are intragranular excipients, and the lubricant/glidant is an extragranular excipient.

Further preferably, the method according to the above, wherein the weight ratio of the drug-containing immediate-release shell to the drug-containing delayed-release core is at least 4, preferably from 4 to 12 or 350:60.

Further preferably, the method according to the above, wherein the weight gain of the gastric-soluble coating of the shell is 2% to 5%, preferably 3%.

Further preferably, the method according to the above, wherein the filler is selected from the group consisting of microcrystalline cellulose (MCC), lactose, pregelatinized starch, starch, dibasic calcium phosphate, mannitol, dextrin, and combinations thereof;
the binder is selected from the group consisting of povidone (PVP), copovidone, hydroxypropyl cellulose (HPC), hypromellose (HPMC), and combinations thereof;
the disintegrant is selected from the group consisting of starch, pregelatinized starch, low-substituted hydroxypropyl cellulose (L-HPC), sodium starch glycolate (SSG), crospovidone (PVPP), croscarmellose sodium (CCS), and combinations thereof;
the sustained-release material is selected from the group consisting of hydroxypropyl cellulose (HPC), hypromellose (HPMC), alginate, ethylcellulose (EC), polyvinyl acetate (PVA), xanthan gum, guar gum, crospovidone (PVPP), glyceryl behenate, and combinations thereof,
the antioxidant is selected from the group consisting of vitamin E (VE), p-hydroxyanisole, butylated hydroxyanisole (BHA), butylated hydroxytoluene (BHT), sodium bisulfite (SBS), sodium metabisulfite (SMBS), propyl gallate (PG), ascorbic acid (VC), and combinations thereof;
the lubricant/glidant is selected from the group consisting of magnesium stearate, sodium stearyl fumarate (SSF), colloidal silicon dioxide, sodium lauryl sulfate (SLS), talc, and combinations thereof.

The present invention provides a method for treating a sleep disorder by orally administering a modified pharmaceutical composition containing a melatonin receptor agonist to a patient. Through research on several aspects, including tablet structure, formulation, and tableting process, the present invention provides a tablet formulation that allows staged release of the active pharmaceutical ingredient, and a preparation process thereof, which are particularly suitable for sleep-regulating drugs. The present invention addresses the lack of such tablets in the prior art.

In a dissolution test of the tablet provided by the present disclosure, results were as shown in FIGS. 2, 4, and 5. The release of the drug-containing immediate-release shell is completed within 10-30 minutes. Due to the protection of the drug-free enteric coating, no drug is released within a period of time, such as 1.5-4 hours. Then a second stage is started, and the drug in the drug-containing delayed-release tablet core is released to realize a relatively ideal two-stage release mode. The tablet is used in a sleep-regulating drug and may quickly take effect after being taken to help fall asleep quickly. The second-stage release is started after a period of time, such that early awakening is prevented and the total sleep time is increased.

In vivo plasma concentration data show that,

In order to obtain the tablet realizing the release mode, the present disclosure performs drug-free stomach-soluble coating and drug-free enteric coating on the drug-containing delayed-release tablet core in the tablet structure. In a research and development test of the present disclosure, since the enteric coating technology requires a relatively long time, the tablet core is likely to be broken easily in the enteric coating process. Therefore, preferably, the stomach-soluble coating may be performed to protect the tablet core before the enteric coating, and does not delay the release of the tablet core in an enteric environment.

In addition, the present disclosure studies an effect of a weight gaining thickness of the enteric coating on the release mode. It is found that if the enteric coating film is too thin, the tablet core cannot be prevented from releasing in a gastric acid environment. If the coating film is too thick, a process cost will be increased, and the release of the tablet core is delayed. It is found that an ideal weight-gaining range of the enteric coating is 4-12%; When it is below 4%, the desired dual-stage release is difficult to achieve, as shown in Comparative Example 3 and FIG. 15.

The study of the present disclosure also finds that since some enteric coating materials are not tough enough, the enteric coating is easy to crack or generate gaps in a tableting process so as to cause an early release of the delayed-release tablet core, or a part of the enteric coating materials are not suitable for a film-forming material and it may only ensure that the enteric coating materials are not dissolved below a pH of 4.0. For example, as shown in FIG. 7, when the coating material is Opadry enteric 940680000 and 910610002, the tablet core is released in an environment of a pH of 5.0, and a dual-stage release may not be realized.

In order to obtain the tablet that achieves a targeted release mode of the present disclosure, the present study finds that when the film-forming material of the selected enteric coating material is one or more materials from the following group: acrylic resin I, II, and III, cellulose acetate phthalate (CAP), hydroxypropyl methylcellulose phthalate (HPMCP), polyvinyl acetate phthalate (PVAP), cellulose acetate trimellitate (CAT), acrylic resin EuS100 (Eudragit S100) and EuL100 (Eudragit L100), a methacrylic acid-methyl methacrylate copolymer, a methacrylic acid-ethyl acrylate copolymer (Eudragit L30D-55), etc. Preferably, the methacrylic acid-ethyl acrylate copolymer is used. For example, when Eudragit L100-55 (Eudragit L30D-55) is selected, the prepared tablet may achieve an ideal release effect.

The present invention also investigated the weight ratio between the immediate-release outer shell and the delayed-release tablet core. It was found that an immediate-release outer shell containing the drug and having an appropriate thickness can effectively prevent cracking of the enteric coating caused by compression during the manufacturing process. The present invention ensures that the immediate-release outer shell achieves the desired thickness by limiting the weight ratio of the drug-containing immediate-release outer shell to the drug-containing sustained-release tablet core to be from 4:1 to 12:1. The experimental data from Examples 7-9 and Comparative Examples 4 and 5 demonstrate that when the weight ratio of the drug-containing immediate-release outer shell to the drug-containing sustained-release tablet core is less than 4 (Comparative Example 4), the desired two-stage release profile cannot be achieved (FIG. 16). When the ratio exceeds 12 (e.g., Comparative Example 5, with a ratio of 14), under the condition of a constant active pharmaceutical ingredient dose, the tablet diameter exceeds 13 mm. Such large-sized tablets are difficult to swallow, reduce patient compliance, and increase manufacturing costs, and therefore represent a less preferred option. When the ratio is controlled within the range of 4 to 12 (Examples 1-3, 7, 8, 9), the desired two-stage release characteristics are achieved. See the in vitro dissolution data shown in FIGS. 2, 4, 5, and 16.

The study of the present disclosure also finds that certain parameters in the preparation process affect the yield of the tablet of the present disclosure and whether the tablet reaches a targeted release mode, thereby affecting the practicability/promotable value of the technology of the present disclosure, such as the pre-pressing pressure and the main pressure of the tablet pressing in step (3) of the preparation method: when the pre-pressing pressure is more than 1.5 kN, such as 2 kN, most tablet core enteric coatings are damaged, the tablet core of the obtained tablet is released in an acid, and when the pre-pressing pressure is 0.02-1.5 kN, wrapping by the tablet core enteric coatings is complete and the tablet core is not released in an acid for 4 h, contributing to obtain an ideal release mode; when the main pressure lower than 15 kN, such as 10 kN, is used for tableting, the pressed tablet is relatively soft and easy to break, and the friability is not qualified; and when the main pressure is greater than 35 kN, the pressed tablet has a top crack phenomenon and an enteric coating film of the tablet core is cracked, such that an active drug in the tablet core is released in the acid. A test shows that when the main pressure is within a range of 15-35 kN, the friability is qualified, wrapping by the tablet core enteric coatings is complete, and the tablet core is not released in the acid for 4 h.

When the medicinally active substance is a sleep-regulating drug, the test of the present disclosure shows that when a content ratio of a pharmaceutically effective substance in the drug-containing immediate-release shell to the drug-containing delayed-release tablet core is (1-3):1, an ideal blood concentration state may be realized. If the ratio of the drug in the drug-containing delayed-release tablet core is too high, a patient is not easy to wake up in a later period. If the ratio of the drug in the drug-containing delayed-release tablet core is too low and the ratio in the immediate-release shell is too high, an increase of the blood concentration is too high and too fast, the blood concentration in the later stage is low, and a blood concentration fluctuation is too large.

### BRIEF DESCRIPTION OF THE DRAWINGS

FIG. 1 is a cross-sectional structural schematic diagram of the tablet of the present disclosure,
wherein 1—drug-containing delayed-release tablet core, 2—drug-free stomach-soluble coating, 3—drug-free enteric coating, 4—drug-containing immediate-release shell, and 5—shell stomach-soluble coating;

FIG. 2 shows a result of a dissolution test of the tablet obtained in example 1;

FIG. 3 shows a result of a dissolution test of the tablet obtained in Comparative Example 1;

FIG. 4 shows a result of a dissolution test of the tablet obtained in example 2;

FIG. 5 shows a result of a dissolution test of the tablet obtained in example 3;

FIG. 6 shows an effect of a pre-pressing pressure on a tablet drug release model in a preparation process of the tablet of the present disclosure;

FIG. 7 shows an effect of a main-pressing pressure on a tablet drug release model in a preparation process of the tablet of the present disclosure;

FIG. 8 shows an effect of an enteric coating material on a drug release model of the tablet of the present disclosure;

FIG. 9 shows an effect of an enteric coating material weight gain on a drug release model of the tablet of the present disclosure; and

FIG. 10 shows an in-vivo release data comparison of the tablet and the reference preparation of the present disclosure.

FIG. 11 shows a result of an in vivo release test of the tablet obtained in example 4;

FIG. 12 shows a result of an in vivo release test of the tablet obtained in example 5;

FIG. 13 shows a result of an in vivo release test of the tablet obtained in example 6;

FIG. 14 shows a result of an in vivo release test of the tablet obtained in Comparative Example 2;

FIG. 15 shows a result of an in vivo release test of the tablet obtained in Comparative Example 3;

FIG. 16 shows results of in vitro dissolution tests of the tablets obtained in examples 7-9 and Comparative Examples 4-5;

### DETAILED DESCRIPTION OF THE EMBODIMENTS

In order to make the objective, technical solutions and advantages of the present application clearer, the technical solutions in the examples of the present application will be described in more detail below with reference to the drawings in the examples of the present application.

As shown in FIG. 1:
1. A tablet allowing release by stages of a drug, wherein the tablet contains a drug-containing delayed-release tablet core (1), a drug-free enteric coating (3), a drug-containing immediate-release shell (4), and a shell stomach-soluble coating (5) sequentially from inside to outside.
2. Preferably, in the tablet, a drug-free stomach-soluble coating (2) is arranged between the drug-containing delayed-release tablet core (1) and the drug-free enteric coating (3).
3. Preferably, in the tablet, a difference between the diameter of the tablet and the diameter of the drug-containing delayed-release tablet core (1) is ≥2 mm.
4. Preferably, in the tablet, a difference between the diameter of the tablet and the diameter of the drug-containing delayed-release tablet core (1) is between 2-8 mm or 2-6 mm or 3-7 mm or 4-8 mm.
5. The tablet according to claim 1, a difference between the thickness of the tablet and the thickness of the drug-containing delayed-release tablet core (1) is ≥1 mm.
6. Preferably, in the tablet, a difference between the thickness of the tablet and the thickness of the drug-containing delayed-release tablet core (1) is between 1-8 mm or 1.5-6 mm or 2-6 mm.
7. Preferably, in the tablet, the diameter of the tablet is 5-13 mm or 9.9-10.25 mm; and the diameter of the drug-containing delayed-release tablet core (1) is 3-9 mm or 4.9-5.25 mm.
8. Preferably, in the tablet,
the thickness of a narrower side from an upper surface of the drug-containing delayed-release tablet core (1) to an upper surface of the tablet is ≥0.5 mm; and
the thickness of a narrower side between an outer edge of the drug-containing delayed-release tablet core (1) and an outer edge of the tablet is ≥0.5 mm.
9. Preferably, in the tablet, in a longitudinal section direction of the tablet, the drug-containing delayed-release tablet core (1) is located in a geometrically central region of the tablet; and
in a cross section direction of the tablet, the drug-containing delayed-release tablet core (1) is located in a geometrically central region of the tablet.
10. Preferably, in the tablet, the drug refers to a sleep-regulating drug, such as ramelteon.

In an example of a typical tablet of the present disclosure, the tablet core is pressed using a punching die having a diameter of 5 mm, such that the theoretical diameter of the drug-containing tablet core in an uncoated state should be 5 mm. The whole tablet is pressed using a punching die having a diameter of 10 mm, such that the theoretical diameter of the whole tablet in an uncoated state is 10 mm. The tablet core is theoretically in the middle of the whole tablet, such that theoretically, but actually, a slight error exists. A vertical distance from a periphery of the tablet core to a periphery of the tablet does not affect a final release curve within a range of 0.5-4.5 mm.

### Example 1 Preparation of Tablet (Delayed-Release Tablet Core is Immediate-Release)

| Preparation composition | | Example 1 | |
|---|---|---|---|
| | | Prescription | Prescription dosage |
| | | ratio % | per unit mg |
| Delayed-release | Drug: Ramelteon | 6.67 | 4 |
| tablet core | Filler Lactose | 68.81 | 41.29 |
| | Filler MCC | 15 | 9 |
| | Binder PVP/VA | 2.5 | 1.5 |
| | Disintegrant CCMC-Na | 6 | 3.6 |
| | Antioxidant BHT | 0.02 | 0.012 |
| | Lubricant/flow aid | 1 | 0.6 |
| | (magnesium stearate) | | |
| | Total | 100 | 60 |
| | | Weight gain | Prescription dosage |
| | | ratio % | per unit mg |
| Tablet core | Opadry ® 85F38197-CN | 2 | 1.2 |
| coating | Eudragit L 30D-55 | 6 | 3.67 |
| | | Prescription | Prescription dosage |
| | | ratio % | per unit mg |
| Immediate-release | Ramelteon | 1.14 | 4 |
| outer shell | Lactose | 74.34 | 260.18 |
| | MCC | 15 | 52.5 |
| | PVP/VA | 2.5 | 8.75 |
| | CCMC-Na | 6 | 21 |
| | BHT | 0.02 | 0.07 |
| | Magnesium stearate | 1 | 3.5 |
| | Total | 100 | 350 |
| | | Weight gain | Prescription dosage |
| | | ratio % | per unit mg |
| Coating | OpadryOpadry ® 85F38197-CN | 3 | 12.45 |

| | | |
|---|---|---|
| Total | / | 427.32 |

A preparation method was as follows:
(1) an immediate-release composition was prepared, sieved internal auxiliary materials (lactose, MCC, and PVP/VA) were weighed according to the prescription dosage, ramelteon and BHT were added into a certain amount of ethanol to prepare a solution, the solution was subjected to a wet granulation, the granule was dried, sieved CCMC-Na and magnesium stearate were added, and the materials were uniformly mixed;
(2) a delayed-release composition was prepared, ramelteon was weighed according to the prescription dosage of a main drug, sieved auxiliary materials were weighed according to an auxiliary material formula, and the materials were fully and uniformly mixed according to an equal amount adding method;
(3) a tablet core was pressed by using a delayed-release preparation composition and subjected to stomach-soluble coating and enteric coating; and
(4) an immediate-release preparation composition was used as a shell and the delayed-release tablet core was used as a tablet core to press a core-wrapped tablet, and the immediate-release composition was used as a first layer to be filled into a middle die; the delayed-release tablet core was placed on the first layer and pre-pressed (0.02 kN); and the immediate-release composition was filled again as a third layer and pressed into a tablet with a main pressure of 20 kN.

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 7.2, and determination at 210 nm by using an ultraviolet (UV) spectrophotometry.

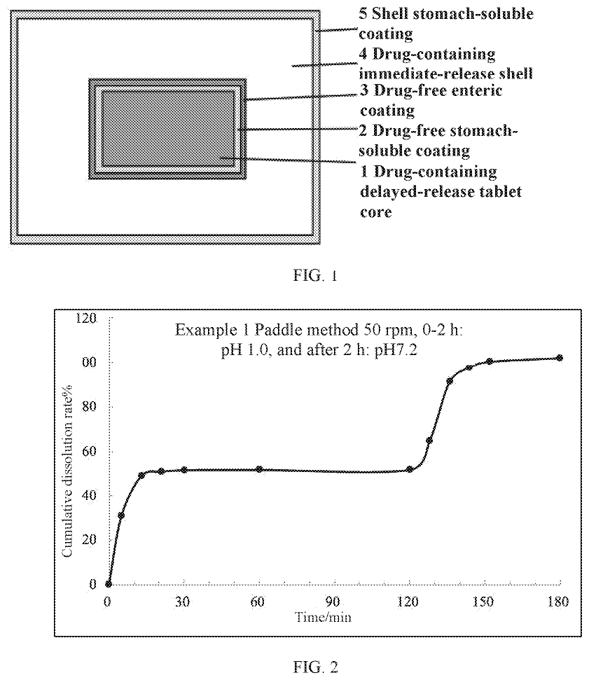

A test result of a dissolution rate was shown in FIG. 2 that the immediate-release shell was completely released within 15 minutes; and no drug was released during a period from 15 minutes later to 120 minutes earlier, and the delayed-release tablet core was completely released within 30 minutes after 120 minutes.

### Comparative Example 1 Preparation of Tablet (Delayed-Release Tablet Core is Slow-Release)

| Preparation composition | | Comparative Example 1 | |
|---|---|---|---|
| | | Prescription | Prescription dosage |
| | | ratio % | per unit mg |
| Delayed-release | Ramelteon | 10.00 | 6 |
| tablet core | Filler Calcium | 51.45 | 30.87 |
| | hydrogen phosphate | | |
| | Filler MCC | 15.00 | 9.00 |
| | Binder PVP | 2.50 | 1.50 |
| | Slow-release | 20.00 | 12.00 |
| | material HPMC | | |
| | Antioxidant BHA | 0.05 | 0.03 |
| | Lubricant/flow aid | 1.00 | 0.60 |
| | (magnesium stearate) | | |
| | Total | 100.00 | 60.00 |
| | | Weight gain | Prescription dosage |
| | | ratio % | per unit mg |
| Tablet core | Opadry ® 40L180000 | 2.00 | 1.20 |
| coating | Acryl ® 93084720 | 8.00 | 4.90 |
| | | Prescription | Prescription dosage |
| | | ratio % | per unit mg |
| Immediate-release | Ramelteon | 0.57 | 2.00 |
| outer shell | Lactose | 69.38 | 242.83 |
| | MCC | 20.00 | 70.00 |
| | PVP | 3.00 | 10.50 |
| | Crospovidone | 6.00 | 21.00 |
| | BHA | 0.05 | 0.18 |
| | Magnesium stearate | 1.00 | 3.50 |
| | Total | 100.00 | 350.00 |
| | | Weight gain | Prescription dosage |
| | | ratio % | per unit mg |
| Coating | Opadry ® 40L180000 | 3.00 | 12.48 |

| | | |
|---|---|---|
| Total | / | 428.58 |

A preparation method was as follows:
(1) an immediate-release composition was prepared, ramelteon and sieved internal auxiliary materials (lactose, MCC, and BHA) were weighed according to the prescription dosage, PVP was added into a certain amount of water to prepare a binder, a wet granulation was performed, the granule was dried, sieved crospovidone and magnesium stearate were added, and the materials were uniformly mixed;
(2) a delayed-release composition was prepared, ramelteon and sieved internal auxiliary materials (calcium hydrogen phosphate, MCC, HPMC, and BHA) were weighed according to the prescription dosage, PVP was added into a certain amount of water to prepare a binder, a wet granulation was performed, the granule was dried, sieved magnesium stearate was added, and the materials were uniformly mixed;
(3) a tablet core was pressed by using a delayed-release preparation composition and subjected to stomach-soluble coating and enteric coating; and
(4) an immediate-release preparation composition was used as a shell and the delayed-release tablet core was used as a tablet core to press a core-wrapped tablet, and the immediate-release composition was used as a first layer to be filled into a middle die; the delayed-release tablet core was placed on the first layer and pre-pressed (0.04 kN); and the immediate-release composition was filled again as a third layer and pressed into a tablet with a main pressure of 25 kN.

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 6.8, and determination at 210 nm by using an ultraviolet (UV) spectrophotometry.

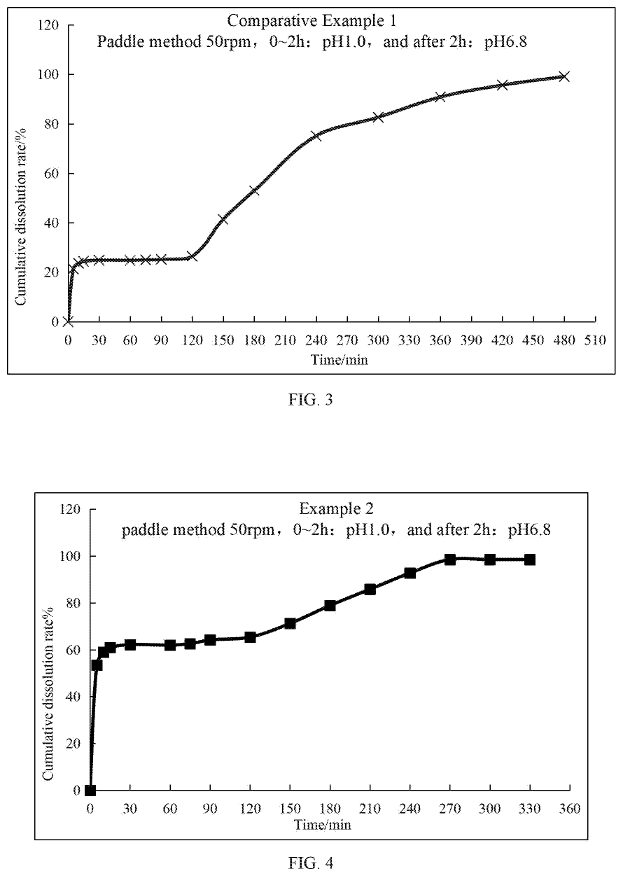

A test result of a dissolution rate was shown in FIG. 3.

The immediate-release shell was completely released within 10 minutes; no drug was released during a period from 15 minutes later to 120 minutes earlier, and the delayed-release tablet core started to release a pharmaceutical ingredient after 120 minutes continuously to the 10th hour. Under the formula, if the ratio of the drug contained in the delayed-release tablet core was too high, a person taking the drug is not easy to wake up in a later period.

### Example 2 Preparation of Tablet (Delayed-Release Tablet Core is Slow-Release)

| Preparation composition | | Example 2 | |
|---|---|---|---|
| | | Prescription | Prescription dosage |
| | | ratio % | per unit mg |
| Delayed-release | Ramelteon | 5.00 | 3.00 |
| tablet core | Filler Mannitol | 44.90 | 26.94 |
| | Filler MCC | 40.00 | 24.00 |
| | Binder PVP/VA | 4.00 | 2.4 |
| | Slow-release material | 5.00 | 3.00 |
| | Sodium alginate | | |
| | Antioxidant VC | 0.10 | 0.06 |
| | Lubricant/flow aid | 0.50 | 0.30 |
| | Colloidal silicon dioxide | | |
| | Lubricant/flow aid | 0.50 | 0.30 |
| | Sodium stearyl fumarate | | |
| | Total | 100.00 | 60.00 |
| | | Weight gain | Prescription dosage |
| | | ratio % | per unit mg |
| Tablet core | Opadry ® 03K170007-CN | 2.00 | 1.20 |
| coating | Eudragit L100 | 7.00 | 4.28 |
| | | Prescription | Prescription dosage |
| | | ratio % | per unit mg |
| Immediate-release | Ramelteon | 1.43 | 5.00 |
| outer shell | MCC | 71.47 | 250.15 |
| | Mannitol | 15.00 | 52.50 |
| | PVP/VA | 3.00 | 10.50 |
| | Sodium carboxymethyl starch | 8.00 | 28.00 |
| | VC | 0.10 | 0.35 |
| | Colloidal silicon dioxide | 0.50 | 1.75 |
| | Sodium stearyl fumarate | 0.50 | 1.75 |
| | Total | 100.00 | 350.00 |
| | | Weight gain | Prescription dosage |
| | | ratio % | per unit mg |
| Coating | Opadry-stomach-soluble | 3.00 | 12.46 |
| | 03K170007-CN | | |

| | | |
|---|---|---|
| Total | / | 427.95 |

A preparation method was as follows:
(1) an immediate-release composition was prepared, ramelteon was weighed according to the prescription dosage of a main drug, sieved auxiliary materials were weighed according to an auxiliary material formula, and the materials were fully and uniformly mixed according to an equal amount adding method;
(2) a delayed-release composition was prepared, ramelteon was weighed according to the prescription dosage, sieved auxiliary materials were weighed according to an auxiliary material formula, and the materials were fully and uniformly mixed according to an equal amount adding method;
(3) a tablet core was pressed by using a delayed-release preparation composition and subjected to stomach-soluble coating and enteric coating; and
(4) an immediate-release preparation composition was used as a shell and the delayed-release tablet core was used as a tablet core to press a core-wrapped tablet, and the immediate-release composition was used as a first layer to be filled into a middle die; the delayed-release tablet core was placed on the first layer and pre-pressed (0.02 kN); and the immediate-release composition was filled again as a third layer and pressed into a tablet with a main pressure of 17 kN.

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 6.8, and determination at 210 nm by using an ultraviolet (UV) spectrophotometry.

A test result of a dissolution rate was shown in FIG. 4.

The immediate-release shell was completely released within 10 minutes; no drug was released during a period from 15 minutes later to 120 minutes earlier; and the delayed-release tablet core started to release a pharmaceutical ingredient after 120 minutes continuously to the 5th hour to reach a release of 100% to reach an ideal release mode.

### Example 3 Preparation of Tablet (Delayed-Release Tablet Core is Slow-Release)

| Preparation composition | | Example 3 | |
|---|---|---|---|
| | | Prescription | Prescription dosage |
| | | ratio % | per unit mg |
| Delayed-release | Ramelteon | 5.83 | 3.50 |
| tablet core | Filler Mannitol | 53.67 | 32.20 |
| | Filler MCC | 25.00 | 15.00 |
| | Binder HPC | 6.00 | 3.60 |
| | Slow-release material HPMC | 8.00 | 4.80 |
| | Antioxidant VC | 0.50 | 0.30 |
| | Lubricant/flow aid Sodium | 1.00 | 0.60 |
| | stearyl fumarate | | |
| | Total | 100.00 | 60.00 |
| | | Weight gain | Prescription dosage |
| | | ratio % | per unit mg |
| Tablet core | Opadry ® 20A99172-CN | 2.00 | 1.20 |
| coating | Acryl ® 93018509 | 5.00 | 3.06 |
| | | Prescription | Prescription dosage |
| | | ratio % | per unit mg |
| Immediate-release | Ramelteon | 1.57 | 5.50 |
| outer shell | MCC | 53.93 | 188.75 |
| | Mannitol | 30.00 | 105.00 |
| | PVP | 5.00 | 17.50 |
| | Sodium carboxymethyl starch | 8.00 | 28.00 |
| | VC | 0.50 | 1.75 |
| | Sodium stearyl fumarate | 1.00 | 3.50 |
| | Total | 100 | 350.00 |
| | | Weight gain | Prescription dosage |
| | | ratio % | per unit mg |
| Coating | Opadry ® 20A99172-CN | 3.00 | 12.43 |

| | | |
|---|---|---|
| Total | / | 426.69 |

A preparation method was as follows:
(1) an immediate-release composition was prepared, sieved internal auxiliary materials (mannitol, MCC, PVP, VC, and sodium carboxymethyl starch) were weighed according to the prescription dosage, ramelteon was added into a certain amount of ethanol to prepare a solution, the solution was subjected to a wet granulation, the granule was dried, sieved sodium stearyl fumarate was added, and the materials were uniformly mixed;
(2) a delayed-release composition was prepared, ramelteon was weighed according to the prescription dosage of a main drug, sieved auxiliary materials were weighed according to an auxiliary material formula, and the materials were fully and uniformly mixed according to an equal amount adding method;
(3) a tablet core was pressed by using a delayed-release preparation composition and subjected to stomach-soluble coating and enteric coating; and
(4) an immediate-release preparation composition was used as a shell and the delayed-release tablet core was used as a tablet core to press a core-wrapped tablet, and the immediate-release composition was used as a first layer to be filled into a middle die; the delayed-release tablet core was placed on the first layer and pre-pressed with a pre-pressing pressure (0.03 kN); and the immediate-release composition was filled again as a third layer and pressed into a tablet with a main pressure of 20 kN.

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 7.2, and determination at 210 nm by using an ultraviolet (UV) spectrophotometry.

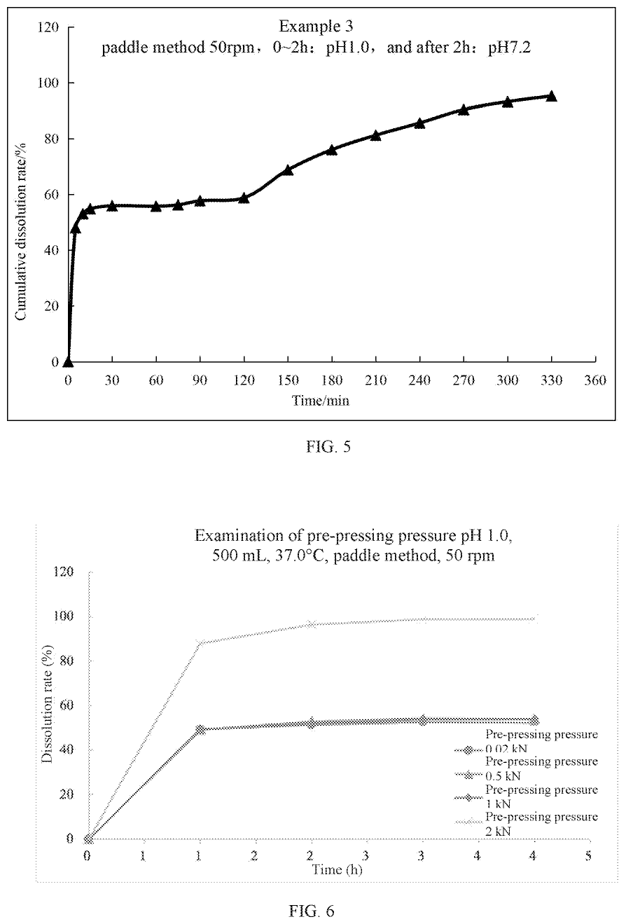

A test result of a dissolution rate was shown in FIG. 5. The immediate-release shell was completely released within 10 minutes; no drug was released during a period from 15 minutes later to 120 minutes earlier; and the delayed-release tablet core started to release a pharmaceutical ingredient after 120 minutes continuously to the 5.5th hour to reach a release of 100% to realize a relatively ideal release mode.

### Test Example 1 Examination of Pre-Pressing Pressure

During the research and development process, it was found that during the core-wrapped tablet pressing process, the pre-pressing pressure may affect an interface interaction and an adhesive force between the tablet core and the immediate-release shell, and may also affect the integrity of the enteric coating film of the tablet core, thereby affecting the dissolution of a sample.

An effect of different pre-pressing pressures on a dissolution effect was investigated using the prescription and the preparation method of example 3.

Different pre-pressing pressures (0.02 kN, 0.5 kN, 1 kN, and 2 kN) were respectively used to perform prepressing; and the immediate-release composition was filled as a third layer again and pressed into a tablet with a main pressure of 20 kN.

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 6.8, and determination at 210 nm by using an ultraviolet (UV) spectrophotometry.

As shown in FIG. 6, when the pre-pressing pressure was 0.02, 0.5, and 1 kN, the wrapping by the tablet core enteric coating was complete, the tablet core was not released in an acid for 4 h, and when the pre-pressing pressure was greater than 1.5 kN such as 2 kN, the wrapping by the tablet core enteric coating was incomplete and damaged, and the tablet core was released in the acid.

### Test Example 2 Examination of Main Pressure

In the research and development process, it was found that when the main pressure was within a range of 15-35 kN, the friability was qualified, wrapping by the tablet core enteric coatings was complete, and the tablet core was not released in an acid for 4 h. The prescription and the tableting process of example 1 were used except that the main pressure was set to be 15, 20, 25, and 35 kN respectively. An effect of the main pressure on a dissolution effect was studied.

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 6.8, and determination at 210 nm by using an ultraviolet (UV) spectrophotometry.

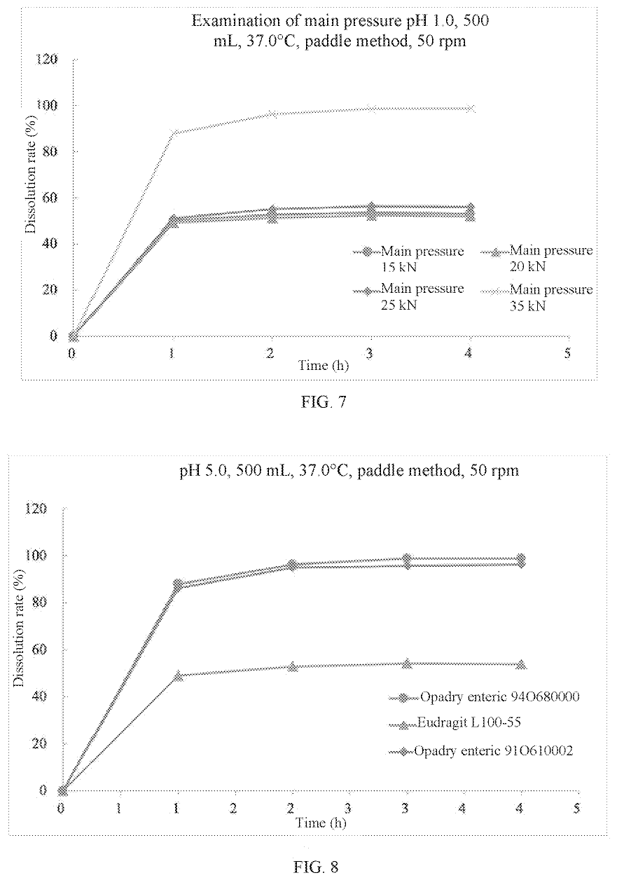

A test result was shown in FIG. 7. When the tablet was pressed with a main pressure of 10 kN, the tablet was cracked and the friability was not qualified. When the main pressure was greater than 35 kN (such as 35 kN), the enteric coating film of the tablet core was cracked, such that an active drug in the tablet core was released in the acid.

### Test Example 3 Comparison of Enteric Coating Material

An effect of different enteric coating materials was compared.

The prescription process of example 3 was used to examine different enteric coating materials respectively.

Dissolution method: a paddle method of 50 rpm, 37° C., and 500 mL of a medium with a pH of 5.0, and determination at 210 nm by using an ultraviolet (UV) spectrophotometry.

A test result was shown in FIG. 8. When the coating material was Opadry enteric 940680000 and 910610002, the enteric coating film may be less tough or may not remain soluble at a pH of 5.0. When Eudragit L100-55 was used as the coating material, a main film-forming material was a methacrylic acid and ethyl acrylate copolymer and an ideal release effect may be achieved.

### Test Example 4 Comparison of Enteric Coating Weight Gain Thickness

The prescription of example 1 was used to study and compare a tableting effect of coating weight gains of 3%, 4%, 5%, 6%, 7%, 8%, 10%, and 12%.

Compared with different enteric coating weight gain thicknesses, when a weight gain range was 4-12%, the dissolution was similar. A second-stage release reached a platform within 0.5-1 h, so as to reach an expected dissolution effect.

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 6.8, and determination at 210 nm by using an ultraviolet (UV) spectrophotometry.

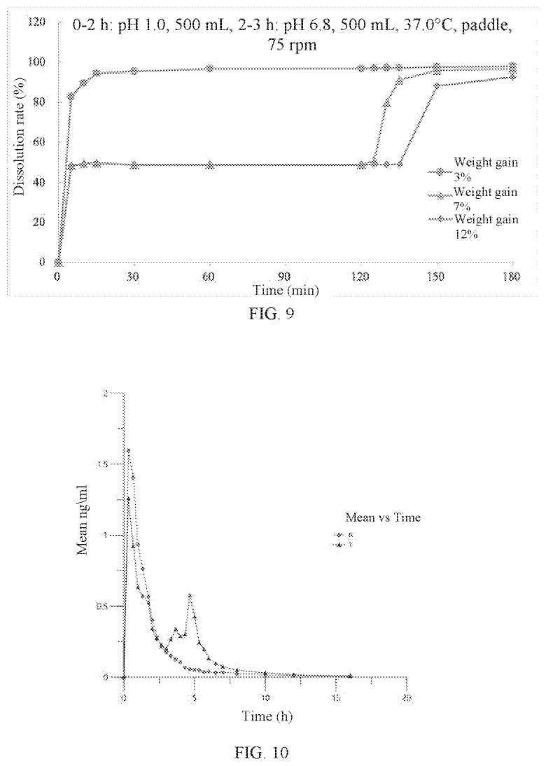

A result was shown in FIG. 9. If the coating film was too thin, the tablet core cannot be protected from releasing in an acid, and an enteric effect cannot be achieved. If the coating film was too thick, a tableting cost was increased.

### Test Example 5 In-Vivo Data

A human body test comparison of a sample (T) prepared in example 1 with a reference preparation (R) was performed, wherein the reference preparation (R) was a ramelteon tablet (Rozerem®) with a specification of 8 mg, a batch number of HH2012, a manufacturer of Japan Takeda Pharmaceuticals, and preservation conditions of shading, sealing, and storing at below 25° C.

A single-center, random, open, two-cycle, double-crossover, and single-dose test design method was used. The dose was 1 tablet/person, a washing period was 2 days, and 8 subjects completed the test.

A test result was shown in FIG. 10. The sample (T) of the present disclosure had a first release Cₘₐₓ smaller than the reference preparation (R), which may reduce the incidence of adverse reactions. Besides, the sample (T) of the present disclosure still had a release at about 5 h compared to the reference preparation (R), which may effectively prevent early awakening.

### Example 4. Tablet of the API Melatonin

| Preparation composition | | Example 4 | |
|---|---|---|---|
| | | Prescription | Prescription dsasge per unit |
| | | ratio % | mg |
| Delayed-release tablet | melatonin | 5.45 | 3.00 |
| core | Lactose | 40.53 | 22.29 |
| | MCC | 40.00 | 22.00 |
| | HPC EF | 3.00 | 1.65 |
| | HPMC | 10.00 | 5.50 |
| | PG | 0.02 | 0.01 |
| | Sodium stearyl fumarate | 1.00 | 0.55 |
| | Total | 100.00 | 55.00 |
| | | Weight gain | Prescription dosage per unit |
| | | ratio % | mg |
| Tablet core Coating | Eudragit L100 | 4.00 | 2.20 |
| | | Prescription | Prescription dosage per unit |
| | | ratio % | mg |
| Immediate-release outer | melatonin | 1.88 | 6.00 |
| shell | Calcium hydrogen | 48.12 | 153.97 |
| | phosphate | | |
| | MCC | 40.00 | 128.00 |
| | HPC EF | 3.00 | 9.60 |
| | CMS-Na | 6.00 | 19.20 |
| | PG | 0.01 | 0.03 |
| | Sodium stearyl fumarate | 1.00 | 3.20 |
| | Total | 100 | 320 |

| | | |
|---|---|---|
| Total | / | 377.2 |

A preparation method was as follows:
(1) an immediate-release composition was prepared, the API was weighed according to the prescription dosage of the main drug, sieved auxiliary materials were weighed according to the auxiliary material formula, and the materials were fully and uniformly mixed according to an equal amount adding method;
(2) a delayed-release composition was prepared, the API was weighed according to the prescription dosage, sieved auxiliary materials were weighed according to the auxiliary material formula, and the materials were fully and uniformly mixed according to an equal amount adding method;
(3) a tablet core was pressed by using a delayed-release preparation composition and subjected to enteric coating; and
(4) an immediate-release preparation composition was used as a shell and the delayed-release tablet core was used as a tablet core to press a core-wrapped tablet, and the immediate-release composition was used as a first layer to be filled into a middle die; the delayed-release tablet core was placed on the first layer and pre-pressed with a pre-pressing pressure (0.02 kN); and the immediate-release composition was filled again as a third layer and pressed into a tablet with a main pressure of 17 kN.

### Example 5. Tablet of the API Agomelatine

| Preparation composition | | Example 5 | |
|---|---|---|---|
| | | Prescription ratio % | Prescription dosage per unit mg |
| Delayed-release tablet core | agomelatine | 7.50 | 6.00 |
| | Lactose | 60.48 | 48.38 |
| | pregelatinized starch | 20.00 | 16.00 |
| | PVP/VA | 3.00 | 2.40 |
| | PPVP | 8.00 | 6.40 |
| | BHA | 0.02 | 0.02 |
| | Magnesium stearate | 1.00 | 0.80 |
| | Total | 100.00 | 80.00 |
| | | Weight gain ratio % | Prescription dosage per unit mg |
| Tablet core Coating | Eudragit L30D55 | 12.00 | 9.60 |
| | | Prescription ratio % | Prescription dosage per unit mg |
| Immediate-release outer | agomelatine | 3.91 | 18.00 |
| shell | MCC | 71.57 | 329.21 |
| | Mannitol | 15.00 | 69.00 |
| | HPC EF | 2.50 | 11.50 |
| | PPVP | 6.00 | 27.60 |
| | BHA | 0.02 | 0.09 |
| | Magnesium stearate | 1.00 | 4.60 |
| | Total | 100.00 | 460.00 |
| Total | | | 549.6 |

The preparation steps were the same as in Example 4.

### Example 6

| Preparation composition | | Example 6 | |
|---|---|---|---|
| | | Prescription ratio % | Prescription dosage per unit mg |
| Delayed-release tablet core | Ramelteon | 6.67 | 4.00 |
| | pregelatinized starch | 54.31 | 32.59 |
| | MCC | 30.00 | 18.00 |
| | PVP/VA | 3.00 | 1.80 |
| | CCMC-Na | 5.00 | 3.00 |
| | BHT | 0.02 | 0.01 |
| | Magnesium stearate | 1.00 | 0.60 |
| | Total | 100.00 | 60.00 |
| | | Weight gain ratio % | Prescription dosage per unit mg |
| Tablet core Coating | Eudragit S100 | 10.00 | 6.00 |
| | | Prescription ratio % | Prescription dosage per unit mg |
| Immediate-release outer | Ramelteon | 1.14 | 4.00 |
| shell | pregelatinized starch | 59.34 | 207.68 |
| | MCC | 30.00 | 105.00 |
| | PVP/VA | 2.50 | 8.75 |
| | CCMC-Na | 6.00 | 21.00 |
| | BHT | 0.02 | 0.07 |
| | Magnesium stearate | 1.00 | 3.50 |
| | Total | 100.00 | 350.00 |

| | |
|---|---|
| Total | 416.00 |

The preparation steps were the same as in Example 4.

Comparative Example 2 (The difference from Example 6 lies in the enteric coating film-forming material.)

| Preparation composition | | Comparative Example 2 | |
|---|---|---|---|
| | | Prescription ratio % | Prescription dosage per unit mg |
| Delayed-release tablet core | Ramelteon | 6.67 | 4.00 |
| | pregelatinized starch | 54.31 | 32.59 |
| | MCC | 30.00 | 18.00 |
| | PVP/VA | 3.00 | 1.80 |
| | CCMC-Na | 5.00 | 3.00 |
| | BHT | 0.02 | 0.01 |
| | Magnesium stearate | 1.00 | 0.60 |
| | Total | 100.00 | 60.00 |
| | | Weight gain ratio % | Prescription dosage per unit mg |
| Tablet core Coating | Eudragit E100 | 10.00 | 6.00 |
| | | Prescription ratio % | Prescription dosage per unit mg |
| Immediate-release outer | Ramelteon | 1.14 | 4.00 |
| shell | pregelatinized starch | 59.34 | 207.68 |
| | MCC | 30.00 | 105.00 |
| | PVP/VA | 2.50 | 8.75 |
| | CCMC-Na | 6.00 | 21.00 |
| | BHT | 0.02 | 0.07 |
| | Magnesium stearate | 1.00 | 3.50 |
| | Total | 100.00 | 350.00 |

| | | |
|---|---|---|
| Total | / | 416.00 |

The preparation steps were the same as in Example 4; The formulation differs from that of Example 6 in the enteric coating film-forming material.

### Comparative Example 3 (the Difference from Example 6 Lies in the Enteric Coating Weight Gain.)

| Preparation composition | | Comparative Example 3 | |
|---|---|---|---|
| | | Prescription ratio % | Prescription dosage per unit mg |
| Delayed-release tablet core | Ramelteon | 6.67 | 4.00 |
| | pregelatinized starch | 54.31 | 32.59 |
| | MCC | 30.00 | 18.00 |
| | PVP/VA | 3.00 | 1.80 |
| | CCMC-Na | 5.00 | 3.00 |
| | BHT | 0.02 | 0.01 |
| | Magnesium stearate | 1.00 | 0.60 |
| | Total | 100.00 | 60.00 |
| | | Weight gain ratio % | Prescription dosage per unit mg |
| Tablet core Coating | Eudragit L100 | 2.00 | 1.20 |
| | | Prescription ratio % | Prescription dosage per unit mg |
| Immediate-release outer | Ramelteon | 1.14 | 4.00 |
| shell | pregelatinized starch | 59.34 | 207.68 |
| | MCC | 30.00 | 105.00 |
| | PVP/VA | 2.50 | 8.75 |
| | CCMC-Na | 6.00 | 21.00 |
| | BHT | 0.02 | 0.07 |
| | Magnesium stearate | 1.00 | 3.50 |
| | Total | 100.00 | 350.00 |
| | | / | 416.00 |

The preparation steps were the same as in Example 4; The formulation differs from that of Example 6 in the enteric coating weight gain.

### In Vivo Data of Examples 4-6 and Comparative Examples 2 and 3

In vivo tests were conducted using the tablets of Examples 4-6 and Comparative Examples 2 and 3, with a single-center, randomized, open-label, two-period, two-sequence crossover, single-dose study design. The dosage was 1 tablet per subject, with a washout period of 2 days. Eight subjects completed the study. The in vivo plasma concentration monitoring data are shown in the following table.

### In Vivo Plasma Concentration Monitoring Data

| | | | | Comparative | Comparative |
|---|---|---|---|---|---|
| | Example 4 | Example 5 | Example 6 | Example 2 | Example 3 |
| Time/h | ng/ml | μg/ml | ng/ml | ng/ml | ng/ml |
| 0 | 0.00 | 0.00 | 0.00 | 0.00 | 0.00 |
| 0.33 | 1.15 | 25.00 | 1.08 | 2.53 | 1.60 |
| 0.67 | 1.01 | 36.00 | 0.31 | 1.68 | 1.41 |
| 1 | 0.83 | 44.00 | 0.17 | 1.39 | 0.94 |
| 1.33 | 0.64 | 37.00 | 0.11 | 0.75 | 0.76 |
| 1.75 | 0.45 | 31.00 | 0.09 | 0.57 | 0.57 |
| 2 | 0.31 | 24.00 | 0.06 | 0.47 | 0.40 |
| 2.33 | 0.26 | 29.00 | 0.05 | 0.51 | 0.28 |
| 2.67 | 0.22 | 32.00 | 0.04 | 0.51 | 0.22 |
| 3 | 0.12 | 38.00 | 0.03 | 0.38 | 0.18 |
| 3.33 | 0.05 | 34.00 | 0.03 | 0.28 | 0.15 |
| 3.67 | 0.07 | 27.00 | 0.02 | 0.24 | 0.12 |
| 4 | 0.15 | 22.00 | 0.02 | 0.20 | 0.11 |
| 4.33 | 0.24 | 18.00 | 0.02 | 0.15 | 0.07 |
| 4.67 | 0.38 | 15.00 | 0.03 | 0.12 | 0.06 |
| 5 | 0.42 | 13.00 | 0.12 | 0.10 | 0.05 |
| 5.33 | 0.44 | 11.00 | 0.26 | 0.16 | 0.05 |
| 5.67 | 0.43 | 9.00 | 0.58 | 0.11 | 0.04 |
| 6 | 0.39 | 8.00 | 0.81 | 0.09 | 0.04 |
| 6.5 | 0.31 | 6.00 | 0.45 | 0.06 | 0.03 |
| 7 | 0.26 | 5.00 | 0.35 | 0.06 | 0.03 |
| 8 | 0.2 | 4.00 | 0.20 | 0.05 | 0.02 |
| 10 | 0.12 | 2.00 | 0.12 | 0.03 | 0.02 |
| 12 | 0.10 | 2.00 | 0.08 | 0.02 | 0.01 |
| 16 | 0.07 | 1.00 | 0.03 | 0.01 | 0.01 |
| 24 | 0.04 | 1.00 | 0.02 | 0.00 | 0.00 |
| peak interval time | 5 | 2 | 5.67 | / | / |
| (h) | | | | | |

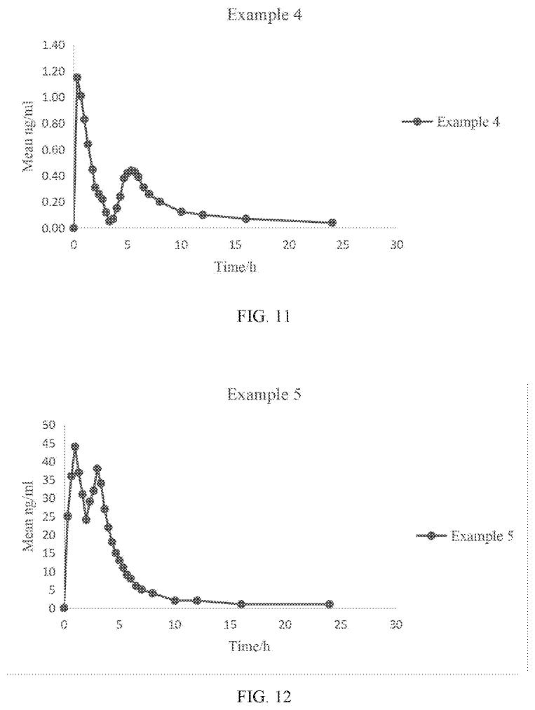

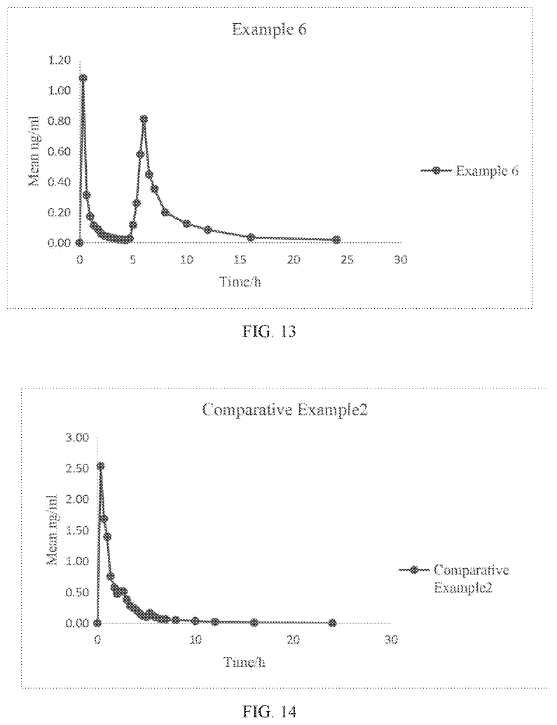

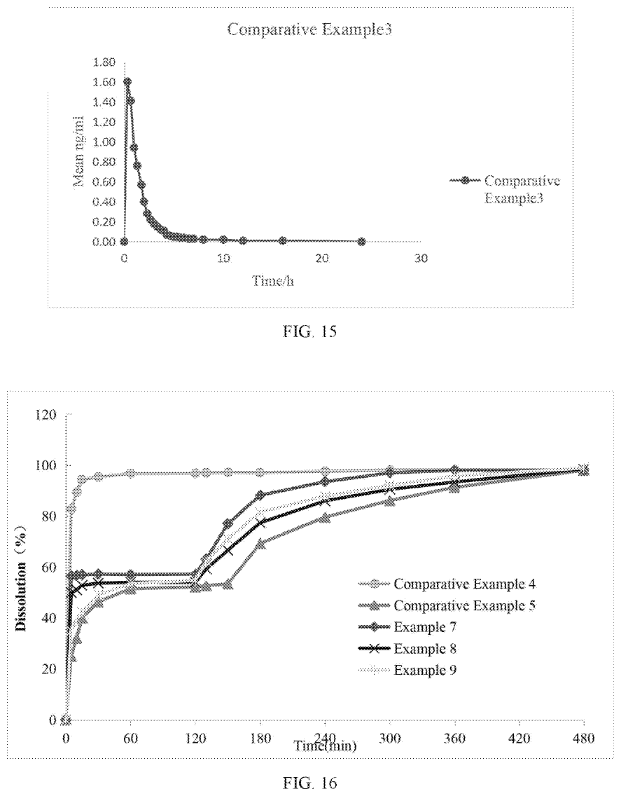

Based on the above test data, curves were plotted, as shown in FIGS. 11-15. The tablets of Examples 4-6 each exhibited two drug concentration peaks in vivo. The time interval between the first peak and the last peak was 0.3 to 10 hours, and the first peak occurred between 0.1 and 3 hours after administration. The first peak promotes rapid sleep onset, and the second peak effectively prevents early awakening.

Comparative Example 2 differs from Example 6 only in the enteric coating film-forming material. When using methacrylic acid ammonio alkyl ester copolymer type E, the comparative formulation failed to produce two plasma concentration peaks in vivo.

Comparative Example 3 differs from Example 6 only in the enteric coating weight gain. In Comparative Example 3, the enteric coating weight gain was less than 4%, specifically only 2%, and the comparative formulation failed to produce two plasma concentration peaks in vivo.

### Example 7. The Weight Ratio of the Immediate-Release Outer Shell to the Delayed-Release Tablet Core is 4:1

| Preparation composition | | Example 7 | |
|---|---|---|---|
| | | | Prescription dosage per |
| | | Prescription ratio % | unit mg |
| Delayed-release | Ramelteon | 5.00 | 3.00 |
| tablet core | Mannitol | 44.90 | 26.94 |
| | MCC | 40.00 | 24.00 |
| | PVP/VA | 4.00 | 2.40 |
| | Sodium alginate | 5.00 | 3.00 |
| | VC | 0.10 | 0.06 |
| | Colloidal silicon dioxide | 0.50 | 0.30 |
| | Sodium stearyl fumarate | 0.50 | 0.30 |
| | Total | 100.00 | 60.00 |
| | | | Prescription dosage per |
| | | Prescription ratio % | unit mg |
| Tablet core | Opadry ® 85F38197-CN | 2.00 | 1.20 |
| Coating | Eudragit L 30D-55 | 7.00 | 4.28 |
| | | | Prescription dosage per |
| | | Prescription ratio % | unit mg |
| Immediate- | Ramelteon | 1.67 | 4.00 |
| release outer shell | MCC | 66.23 | 158.96 |
| | Mannitol | 20.00 | 48.00 |
| | PVP/VA | 3.00 | 7.20 |
| | Sodium carboxymethyl starch | 8.00 | 19.20 |
| | VC | 0.10 | 0.24 |
| | Colloidal silicon dioxide | 0.50 | 1.20 |
| | Sodium stearyl fumarate | 0.50 | 1.20 |
| | Total | 100.00 | 240 |
| | | | Prescription dosage per |
| | | Prescription ratio % | unit mg |
| Coating | Opadry ® 85F38197-CN | 3.00 | 9.16 |
| Total | | / | 314.64 |

The preparation steps were the same as in Example 1.

### Example 8. The Weight Ratio of the Immediate-Release Outer Shell to the Delayed-Release Tablet Core is 8:1

| Preparation composition | | Example 8 | |
|---|---|---|---|
| | | Prescription | Prescription dosage |
| | | ratio % | per unit mg |
| Delayed-release | Ramelteon | 5.83 | 3.50 |
| tablet core | Mannitol | 53.67 | 32.20 |
| | MCC | 25.00 | 15.00 |
| | HPC | 6.00 | 3.60 |
| | HPMC K4M | 8.00 | 4.80 |
| | VC | 0.50 | 0.30 |
| | Sodium stearyl fumarate | 1.00 | 0.60 |
| | Total | 100.00 | 60.00 |
| | | Weight gain | Prescription dosage |
| | | ratio % | per unit mg |
| Tablet core | Opadry ® 85F38197-CN | 2.00 | 1.20 |
| coating | Eudragit L 30D-55 | 5.00 | 3.06 |
| | | Prescription | Prescription dosage |
| | | ratio % | per unit mg |
| Immediate-release | Ramelteon | 0.83 | 4.00 |
| outer shell | MCC | 54.67 | 262.40 |
| | Mannitol | 30.00 | 144.00 |
| | PVP | 5.00 | 24.00 |
| | Sodium carboxymethyl | 8.00 | 38.40 |
| | starch | | |
| | VC | 0.50 | 2.40 |
| | Sodium stearyl fumarate | 1.00 | 4.80 |
| | Total | 100.00 | 480 |
| | | Weight gain | Prescription dosage |
| | | ratio % | per unit mg |
| Coating | Opadry ® 85F38197-CN | 3.00 | 16.33 |

| | | |
|---|---|---|
| Total | / | 560.59 |

The preparation steps were the same as in Example 1.

### Example 9. The Weight Ratio of the Immediate-Release Outer Shell to the Delayed-Release Tablet Core is 12:1

| Preparation composition | | Example 9 | |
|---|---|---|---|
| | | | Prescription dosage per |
| | | Prescription ratio % | unit mg |
| Delayed-release | Ramelteon | 5.83 | 3.50 |
| tablet core | Mannitol | 53.67 | 32.20 |
| | MCC | 25.00 | 15.00 |
| | HPC | 6.00 | 3.60 |
| | HPMC K100LV | 8.00 | 4.80 |
| | VC | 0.50 | 0.30 |
| | Sodium stearyl fumarate | 1.00 | 0.60 |
| | Total | 100.00 | 60.00 |
| | | | Prescription dosage per |
| | | Prescription ratio % | unit mg |
| Tablet core Coating | Opadry ® 85F38197-CN | 2.00 | 1.20 |
| | Eudragit L 30D-55 | 5.00 | 3.06 |
| | | | Prescription dosage per |
| | | Prescription ratio % | unit mg |
| Immediate-release | Ramelteon | 0.56 | 4.00 |
| outer shell | MCC | 54.94 | 395.60 |
| | Mannitol | 30.00 | 216.00 |
| | PVP | 5.00 | 36.00 |
| | Sodium carboxymethyl | 8.00 | 57.60 |
| | starch | | |
| | VC | 0.50 | 3.60 |
| | Sodium stearyl fumarate | 1.00 | 7.20 |
| | Total | 100.00 | 720.00 |
| | | | Prescription dosage per |
| | | Prescription ratio % | unit mg |
| Coating | Opadry ® 85F38197-CN | 3.00 | 23.53 |

| | | |
|---|---|---|
| Total | / | 807.79 |

The preparation steps were the same as in Example 1.

Comparative Example 4. The difference from Example 8 lies in that the weight ratio of the immediate-release outer shell to the delayed-release tablet core is 3:1.

| Preparation composition | | Comparative Example 3 | |
|---|---|---|---|
| | | | Prescription dosage per |
| | | Prescription ratio % | unit mg |
| Delayed-release | Ramelteon | 6.67 | 4 |
| tablet core | Lactose | 68.81 | 41.29 |
| | MCC | 15 | 9 |
| | PVP/VA | 2.5 | 1.5 |
| | CCMC-Na | 6 | 3.6 |
| | BHT | 0.02 | 0.012 |
| | Magnesium stearate | 1 | 0.6 |
| | Total | 100 | 60 |
| | | | Prescription dosage per |
| | | Prescription ratio % | unit mg |
| Tablet core Coating | Opadry ® 85F38197-CN | 2 | 1.2 |
| | Eudragit L 30D-55 | 6 | 3.67 |
| | | | Prescription dosage per |
| | | Prescription ratio % | unit mg |
| Immediate-release | Ramelteon | 2.22 | 4 |
| outer shell | Lactose | 68.26 | 122.86 |
| | MCC | 20 | 36.00 |
| | PVP/VA | 2.5 | 4.50 |
| | CCMC-Na | 6 | 10.80 |
| | BHT | 0.02 | 0.04 |
| | Magnesium stearate | 1 | 1.80 |
| | Total | 100 | 180 |
| | | | Prescription dosage per |
| | | Prescription ratio % | unit mg |
| Coating | Opadry ® 85F38197-CN | 3 | 7.35 |
| Total | | / | 252.22 |

The preparation steps were the same as in Example 1.

Comparative Example 5. The difference from Example 7 lies in that the weight ratio of the immediate-release outer shell to the delayed-release tablet core is 14:1.

| | | Comparative Example 5 | |
|---|---|---|---|
| | | | Prescription dosage per |
| Preparation composition | | Prescription ratio % | unit mg |
| Delayed-release | Ramelteon | 6.67 | 4 |
| tablet core | Calcium hydrogen | 54.78 | 32.87 |
| | phosphate | | |
| | MCC | 15 | 9 |
| | PVP | 2.5 | 1.5 |
| | HPMC | 20 | 12 |
| | BHA | 0.05 | 0.03 |
| | Magnesium stearate | 1 | 0.6 |
| | Total | 100 | 60 |
| | | | Prescription dosage per |
| | | Prescription ratio % | unit mg |
| Tablet core Coating | Opadry ® 85F38197-CN | 2 | 1.2 |
| | Eudragit L 30D-55 | 6 | 3.67 |
| | | | Prescription dosage per |
| | | Prescription ratio % | unit mg |
| Immediate-release | Ramelteon | 0.48 | 4 |
| outer shell | Lactose | 60.00 | 504.03 |
| | MCC | 30 | 252.00 |
| | PVP | 2.5 | 21.00 |
| | Crospovidone | 6 | 50.40 |
| | BHA | 0.02 | 0.17 |
| | Magnesium stearate | 1 | 8.40 |
| | Total | 100 | 840 |
| | | | Prescription dosage per |
| | | Prescription ratio % | unit mg |
| Coating | Opadry ® 85F38197-CN | 3 | 27.15 |
| Total | | | 933.28 |

The preparation steps were the same as in Example 1.

In vitro dissolution test:

Dissolution conditions were as follows: Chinese Pharmacopoeia 2015 Edition Part VI General Rules 0931 Method II, a paddle method of 50 rpm, 37° C., and 500 mL of a medium with a pH of 1.0/a medium with a pH of 6.8, and determination at 210 nm by using an ultraviolet (UV) spectrophotometry.

The test data are shown in the following table and FIG. 16.

| | Comparative | Comparative | | | |
|---|---|---|---|---|---|
| Time/min | Example 4 | Example 5 | Example 7 | Example 8 | Example 9 |
| 0 | 0.00 | 0 | 0.0 | 0 | 0 |
| 5 | 82.9 | 22.0 | 56.5 | 50.0 | 35.0 |
| 10 | 89.5 | 27.1 | 56.7 | 51.0 | 39.5 |
| 15 | 94.4 | 35.0 | 57.0 | 52.8 | 42.3 |
| 30 | 95.4 | 38.4 | 57.2 | 53.7 | 49.1 |
| 60 | 96.7 | 39.5 | 57.1 | 54.0 | 53.5 |
| 120 | 96.8 | 40.2 | 57.2 | 54.0 | 54.8 |
| 130 | 97.0 | 44.5 | 63.1 | 59.2 | 62.2 |
| 150 | 97.2 | 53.4 | 77.0 | 66.6 | 71.0 |
| 180 | 97.1 | 69.3 | 88.2 | 77.4 | 81.6 |
| 240 | 97.6 | 79.6 | 93.5 | 86.0 | 87.8 |
| 300 | 98.1 | 86.1 | 97.0 | 90.5 | 92.1 |
| 360 | 98.1 | 91.3 | 98.0 | 93.4 | 95.7 |
| 480 | 98.1 | 98.0 | 98.1 | 98.5 | 98.9 |

What is claimed is:

1. A treatment method, comprising the following steps: orally administering to a patient in need thereof a modified pharmaceutical composition, wherein API of the modified pharmaceutical composition is selected from the group consisting of a melatonin receptor agonist, a GABA receptor agonist, an orexin receptor antagonist, and pharmaceutically acceptable salts, isomers, and active metabolites thereof;
wherein the melatonin receptor agonist is selected from the group consisting of melatonin, agomelatine, and ramelteon;
wherein the GABA receptor agonist is selected from the group consisting of zolpidem, zopiclone, zaleplon, triazolam, alprazolam, diazepam, estazolam, clonazepam, and didasinine;
wherein the orexin receptor antagonist is selected from the group consisting of suvorexant, daridorexant, and lemborexant;
the treatment method refers to any one of (A1) to (A4):
(A1) a method for treating insomnia;
(A2) a method for alleviating insomnia;
(A3) a method for treating early-morning awakening;
(A4) a sleep regulation method for reducing hangover effects.

2. The treatment method according to claim 1, wherein after administration of the modified pharmaceutical composition, a patient's plasma concentration exhibits 2 to 3 peaks, and a time interval between the first peak and the last peak is 0.3 to 10 hours.

3. The treatment method according to claim 2, wherein the first peak occurs from 0.1 to 3 hours after administration.

4. The treatment method according to claim 1, wherein the modified pharmaceutical composition is a tablet, each tablet comprising, from inside to outside in order, a drug-containing delayed-release core, a drug-free enteric coating, and a drug-containing immediate-release shell.

5. The treatment method according to claim 4, wherein the drug-free enteric coating has a weight gain relative to the drug-containing delayed-release core of at least 4%, and wherein the drug-free enteric coating is soluble at a pH above 5, such that the drug-containing delayed-release core begins to release after a lag period of 0.5 to 4 hours following complete release of the drug-containing immediate-release shell, and is fully released within the subsequent 0.1 to 6 hours.

6. The treatment method according to claim 4, wherein the drug-free enteric coating has a weight gain relative to the drug-containing delayed-release core from 4% to 12%, or 5%, 6%, 7%, 8%, and wherein the drug-free enteric coating is soluble at a pH above 5 such that the drug-containing delayed-release core begins to release after a lag period of 0.5 to 4 hours following complete release of the drug-containing immediate-release shell, and is fully released within the subsequent 0.5 to 4 hours.

7. The treatment method according to claim 4, wherein the tablet further comprises a drug-free gastric-soluble coating between the drug-containing delayed-release core and the drug-free enteric coating, wherein the drug-free gastric-soluble coating has a weight gain of 2% to 5%, 3% or 4%.

8. The treatment method according to claim 4, wherein a film-forming material used in the drug-free enteric coating is selected from the group consisting of Acrylic Resin I, Acrylic Resin II, Acrylic Resin III, cellulose acetate phthalate (CAP), hydroxypropyl methylcellulose phthalate (HPMCP), polyvinyl acetate phthalate (PVAP), cellulose acetate trimellitate (CAT), Eudragit S100, Eudragit L100, methacrylic acid-methyl methacrylate copolymer, and methacrylic acid-ethyl acrylate copolymer.

9. The treatment method according to claim 4, wherein a ratio of an amount of active ingredient in the drug-containing immediate-release shell to an amount of active ingredient in the drug-containing delayed-release core is from 1:1 to 3:1.

10. The treatment method according to claim 4, wherein the drug-containing delayed-release core is an immediate-release formulation; and wherein in the drug-containing delayed-release core, weight percentages of excipients are as follows: filler: 60-90%; disintegrant: 2-10%; binder: 2-6%; antioxidant: 0.01-1%; lubricant/glidant: 0.5-3%; wherein the filler and the antioxidant are intragranular excipients, the lubricant/glidant is an extragranular excipient, and the disintegrant can be either an intragranular excipient or an extragranular excipient.

11. The treatment method according to claim 4, wherein the drug-containing delayed-release core is an immediate-release formulation; and wherein in the drug-containing delayed-release core, weight percentages of excipients are as follows: filler: 60-90%; disintegrant: 4-8%; binder: 2.5-5%; antioxidant: 0.05-1%; lubricant/glidant: 0.5-1.5%; wherein the filler and the antioxidant are intragranular excipients, the lubricant/glidant is an extragranular excipient, and the disintegrant can be either an intragranular excipient or an extragranular excipient.

12. The treatment method according to claim 4, wherein the drug-containing delayed-release core is a sustained-release formulation; and wherein in the drug-containing delayed-release core, weight percentages of excipients are as follows: filler: 60-90%; binder: 2-6%; sustained-release material: 4-25%; antioxidant: 0.01-1%; lubricant/glidant: 0.5-3%; wherein the filler, the binder, and the antioxidant are intragranular excipients, and the lubricant/glidant is an extragranular excipient.

13. The treatment method according to claim 4, wherein the drug-containing delayed-release core is a sustained-release formulation; and wherein in the drug-containing delayed-release core, weight percentages of excipients are as follows: filler: 60-90%; binder: 2.5-5%; sustained-release material: 5-20%; antioxidant: 0.05-1%; lubricant/glidant: 0.5-1.5%; wherein the filler, the binder, and the antioxidant are intragranular excipients, and the lubricant/glidant is an extragranular excipient.

14. The treatment method according to claim 4, wherein a weight ratio of the drug-containing immediate-release shell to the drug-containing delayed-release core is at least 4.

15. The treatment method according to claim 4, wherein a weight ratio of the drug-containing immediate-release shell to the drug-containing delayed-release core is from 4 to 12, or 350:60.

16. The treatment method according to claim 4, wherein the tablet further comprises a gastric-soluble coating shell outside of the drug-containing immediate-release shell, wherein a weight gain of the gastric-soluble coating shell is 2% to 5%, or 3%.

17. The treatment method according to claim 10, wherein the filler is selected from the group consisting of microcrystalline cellulose (MCC), lactose, pregelatinized starch, starch, dibasic calcium phosphate, mannitol, dextrin, and combinations thereof;
the binder is selected from the group consisting of povidone (PVP), copovidone, hydroxypropyl cellulose (HPC), hypromellose (HPMC), and combinations thereof;
the disintegrant is selected from the group consisting of starch, pregelatinized starch, low-substituted hydroxypropyl cellulose (L-HPC), sodium starch glycolate (SSG), crospovidone (PVPP), croscarmellose sodium (CCS), and combinations thereof;
a sustained-release material is selected from the group consisting of hydroxypropyl cellulose (HPC), hypromellose (HPMC), alginate, ethylcellulose (EC), polyvinyl acetate (PVA), xanthan gum, guar gum, crospovidone (PVPP), glyceryl behenate, and combinations thereof;
the antioxidant is selected from the group consisting of vitamin E (VE), p-hydroxyanisole, butylated hydroxyanisole (BHA), butylated hydroxytoluene (BHT), sodium bisulfite (SBS), sodium metabisulfite (SMBS), propyl gallate (PG), ascorbic acid (VC), and combinations thereof;
the lubricant/glidant is selected from the group consisting of magnesium stearate, sodium stearyl fumarate (SSF), colloidal silicon dioxide, sodium lauryl sulfate (SLS), talc, and combinations thereof.

18. The treatment method according to claim 11, wherein the filler is selected from the group consisting of microcrystalline cellulose (MCC), lactose, pregelatinized starch, starch, dibasic calcium phosphate, mannitol, dextrin, and combinations thereof;
the binder is selected from the group consisting of povidone (PVP), copovidone, hydroxypropyl cellulose (HPC), hypromellose (HPMC), and combinations thereof;
the disintegrant is selected from the group consisting of starch, pregelatinized starch, low-substituted hydroxypropyl cellulose (L-HPC), sodium starch glycolate (SSG), crospovidone (PVPP), croscarmellose sodium (CCS), and combinations thereof;
a sustained-release material is selected from the group consisting of hydroxypropyl cellulose (HPC), hypromellose (HPMC), alginate, ethylcellulose (EC), polyvinyl acetate (PVA), xanthan gum, guar gum, crospovidone (PVPP), glyceryl behenate, and combinations thereof;
the antioxidant is selected from the group consisting of vitamin E (VE), p-hydroxyanisole, butylated hydroxyanisole (BHA), butylated hydroxytoluene (BHT), sodium bisulfite (SBS), sodium metabisulfite (SMBS), propyl gallate (PG), ascorbic acid (VC), and combinations thereof;
the lubricant/glidant is selected from the group consisting of magnesium stearate, sodium stearyl fumarate (SSF), colloidal silicon dioxide, sodium lauryl sulfate (SLS), talc, and combinations thereof.

19. The treatment method according to claim 12, wherein the filler is selected from the group consisting of microcrystalline cellulose (MCC), lactose, pregelatinized starch, starch, dibasic calcium phosphate, mannitol, dextrin, and combinations thereof;
the binder is selected from the group consisting of povidone (PVP), copovidone, hydroxypropyl cellulose (HPC), hypromellose (HPMC), and combinations thereof;
a disintegrant is selected from the group consisting of starch, pregelatinized starch, low-substituted hydroxypropyl cellulose (L-HPC), sodium starch glycolate (SSG), crospovidone (PVPP), croscarmellose sodium (CCS), and combinations thereof;
the sustained-release material is selected from the group consisting of hydroxypropyl cellulose (HPC), hypromellose (HPMC), alginate, ethylcellulose (EC), polyvinyl acetate (PVA), xanthan gum, guar gum, crospovidone (PVPP), glyceryl behenate, and combinations thereof;
the antioxidant is selected from the group consisting of vitamin E (VE), p-hydroxyanisole, butylated hydroxyanisole (BHA), butylated hydroxytoluene (BHT), sodium bisulfite (SBS), sodium metabisulfite (SMBS), propyl gallate (PG), ascorbic acid (VC), and combinations thereof;
the lubricant/glidant is selected from the group consisting of magnesium stearate, sodium stearyl fumarate (SSF), colloidal silicon dioxide, sodium lauryl sulfate (SLS), talc, and combinations thereof.

20. The treatment method according to claim 13, wherein the filler is selected from the group consisting of microcrystalline cellulose (MCC), lactose, pregelatinized starch, starch, dibasic calcium phosphate, mannitol, dextrin, and combinations thereof;
the binder is selected from the group consisting of povidone (PVP), copovidone, hydroxypropyl cellulose (HPC), hypromellose (HPMC), and combinations thereof;
a disintegrant is selected from the group consisting of starch, pregelatinized starch, low-substituted hydroxypropyl cellulose (L-HPC), sodium starch glycolate (SSG), crospovidone (PVPP), croscarmellose sodium (CCS), and combinations thereof;
the sustained-release material is selected from the group consisting of hydroxypropyl cellulose (HPC), hypromellose (HPMC), alginate, ethylcellulose (EC), polyvinyl acetate (PVA), xanthan gum, guar gum, crospovidone (PVPP), glyceryl behenate, and combinations thereof;
the antioxidant is selected from the group consisting of vitamin E (VE), p-hydroxyanisole, butylated hydroxyanisole (BHA), butylated hydroxytoluene (BHT), sodium bisulfite (SBS), sodium metabisulfite (SMBS), propyl gallate (PG), ascorbic acid (VC), and combinations thereof;
the lubricant/glidant is selected from the group consisting of magnesium stearate, sodium stearyl fumarate (SSF), colloidal silicon dioxide, sodium lauryl sulfate (SLS), talc, and combinations thereof.
